(19) United States
(12) Patent Application Publication
(10) Pub. No.: US 2011/0092898 A1
(43) Pub. Date: Apr. 21, 2011

(54) MEDICINE INJECTION INTO SKIN WITH HEATER CHIP

(75) Inventor: Ming Yeh CHUANG, McKinney, TX (US)

(73) Assignee: TEXAS INSTRUMENTS INCORPORATED, Dallas, TX (US)

(21) Appl. No.: 12/603,118

(22) Filed: Oct. 21, 2009

(51) Int. Cl. A61M 5/30; A61B 5/00

(52) U.S. Cl. 604/68; 600/301

(57) ABSTRACT

A device configured for drug delivery through the skin of a subject, the device comprising a substrate, a proximal side of which is positioned toward the skin of the subject during use; at least one fluid receptacle positioned on the substrate and containing a store of the drug; at least one ejection chamber positioned on the proximal side of the substrate; at least one fluid via fluidly connecting the at least one fluid receptacle with the at least one ejection chamber; a heating element positioned adjacent at least a portion of the at least one ejection chamber, said heating element configured to rapidly heat and vaporize the fluid along that portion of the ejection chamber, causing ejection of fluid therefrom; and logic, wherein the logic is programmable to initiate heating by the heating element.

### TECHNICAL FIELD

This disclosure relates generally to apparatus and methods for subcutaneous fluid delivery. More particularly, this disclosure relates to such apparatus and methods utilizing ink jet type dispensers to precisely introduce a desired fluid(s) into a subject via the skin. Still more particularly, disclosed embodiments utilize a heater chip-type device to introduce one or more desired fluids into the skin, blood and/or muscle of a subject via the skin.

### BACKGROUND

Typical tools to deliver medicine into skin are bulky, the amount of medicine delivered is often not precise, and usage of such tools is sometimes painful. For example, conventional subcutaneous drug delivery is accomplished via needle injection and pressure gun injection. Needle injection may be painful and/or may be feared by patients (e.g., by children and some adults). Needle injection also requires piercing of the skin, which involves a concomitant risk of contamination and possible infection due to contaminants which may be present on the skin.

It has been recognized that medication and other substances may be injected beneath the skin of humans or animals, without the use of a needle, via utilization of a high pressure jet. Hypodermic jet injectors capable of needleless injection have achieved patient acceptance because a needle is not required and because the injection typically causes little or no pain.

Pressure gun injection requires the use of a pressure gun or jet injector, which may be unwieldy and, if not utilized properly, may provide ineffective drug delivery and/or loss of materials. Jet injectors use air pressure rather than needles to deliver vaccine as a fine stream of fluid that passes through the skin and into the tissue. New and improved jet injectors overcome many of the risks associated with injections in low-resource settings.

In the United States and other developed countries, injections are given by trained personnel who always use a new, sterile syringe readily obtained from a nearby, reliably stocked supply room. But in developing countries, the very injections meant to prevent or treat disease can end up causing it instead. For example, injections become dangerous when health workers reuse syringes and needles or lack the training they need to properly administer injections. In addition, systems for disposing of medical waste may be inadequate and used needles may be retrieved from the garbage and resold or may find their way into the hands of children.

At least 50 percent of injections given in developing countries are unsafe, putting people at risk of infection, such as HIV and hepatitis. Elimination of the use of needles for the administration of drugs to patients would help in protecting both patients and health care workers.

The jet injector devices which have been marketed have generally been large, expensive units adapted to retain large quantities of medicament for repeated injections. Most of these machines are not portable and are used chiefly for mass inoculation programs. Such machines are not adapted to self-administration use by the patient. Such cumbersome and expensive units have meant that the use of high pressure jet-injecting devices have been limited. Patents which describe multi-dose jet injectors include U.S. Pat. Nos. 2,785,678; 3,330,276; 3,521,633; 3,908,651; 4,059,107; 4,447; and 4,560,377.

The prior art has also developed some relatively small jet injector devices which are powered by compressed gas. However, these devices have also generally been expensive and difficult to use. Further, as the gas supply is consumed, the gas driving force changes and the gas is subjected to pressure fluctuations caused by changes in the ambient atmosphere temperature. Patents which describe such gas powered injectors include U.S. Pat. Nos. 3,115,133; 3,527,212; 3,688,765; 3,853,125; and 3,945,379. Such gas powered devices have not achieved significant commercial success.

Further, the prior art discloses a number of spring powered jet injectors. While these prior art devices overcome some of the problems encountered with the multi-dose injectors and the gas powered injectors, the prior art spring powered injectors rely on complex filling techniques or large difficult-to-use cartridges from which the medicine is dispensed. Examples of the spring powered injectors include U.S. Pat. Nos. 2,380,534; 2,722,931; 2,762,370; 3,131,692; 3,557,784; and 3,782,380. These devices have not achieved significant commercial success.

Accordingly, there is a need in the art for apparatus and methods of subcutaneously and/or intramuscularly delivering medicines which provide for more precise drug delivery, more patient-friendly drug delivery, programmable drug delivery, and/or more cost-effective drug delivery than conventional methods.

### SUMMARY

Herein disclosed is a device configured for drug delivery through the skin of a subject, the device comprising: a substrate, a proximal side of which is positioned toward the skin of the subject during use; at least one fluid receptacle positioned on the substrate and containing a store of the drug; at least one ejection chamber positioned on the proximal side of the substrate; at least one fluid via fluidly connecting the at least one fluid receptacle with the at least one ejection chamber; a heating element positioned adjacent at least a portion of the at least one ejection chamber, said heating element configured to rapidly heat and vaporize the fluid along that portion of the ejection chamber, causing ejection of fluid therefrom; and logic, wherein the logic is programmable to initiate heating by the heating element, whereby an amount of fluid is ejected from the at least one ejection chamber at a pressure sufficient to penetrate the epidermis of the subject. In embodiments, the logic is programmed to initiate high pressure ejection of an amount of fluid from the at least one ejection chamber according to a preset timer. The substrate may comprise silicon.

In embodiments, the device further comprises means for sensing a biological parameter of the subject and the logic is operable to determine the timing of fluid ejection, the amount of fluid ejection, or both from the at least one ejection chamber based on the sensed biological parameter. In embodiments, the means for sensing comprises a biosensor positioned on the device such that it contacts the surface of the skin of the subject during use. The biological parameter may be selected from the group consisting of the body temperature of the subject, the heart rate of the subject, the blood pressure of the subject, and combinations thereof. In embodiments, the means for sensing comprises a receiver for the value of a biological parameter determined outside the device. The value of the biological parameter may be measured by an external biosensor located outside the device. The external biosensor may be configured to determine a biological parameter upon introduction into the blood of the subject. The external biosensor may be configured to measure the level of at least one component in the blood of the subject. In embodiments, the external biosensor is configured to measure at least one component selected from the group consisting of blood sugar, a medicine the subject is using, and a blood gas.

In embodiments, the drug is selected from the group consisting of insulin, sugar solutions, antidepressants, cholesterol medicines, high blood pressure medicines, and combinations thereof.

Also disclosed herein is a device configured for delivery of fluid through the skin of a subject, the device comprising: a substrate, a proximal side of which is positioned toward the skin of the subject during use; at least one fluid receptacle positioned on the substrate and containing a store of the fluid; at least one ejection chamber positioned on the proximal side of the substrate; at least one fluid via fluidly connecting the at least one fluid receptacle with the at least one ejection chamber; means for causing ejection of fluid from the at least one ejection chamber; and logic, wherein the logic is programmed to initiate high pressure ejection of an amount of fluid from the at least one ejection chamber by the means for causing ejection, the high pressure ejection at a pressure sufficient to penetrate the epidermis of the subject. In embodiments, the means for causing ejection of fluid from the at least one ejection chamber comprises at least one heater or heater array adjacent at least a portion of the at least one ejection chamber. The at least one heater array may comprise a plurality of thin film resistors. In embodiments, the means for causing ejection of fluid from the at least one ejection chamber comprises a transducer selected from the group consisting of piezoelectric, electrorestrictive, magnetostrictive and electromechanical transducers. In embodiments, the fluid is selected from medicines and tattoo dyes.

Also disclosed herein is a method of introducing a fluid into the skin of a subject, the method comprising: positioning a device configured and programmed for subcutaneous fluid delivery in direct contact with the skin of the subject, wherein the device comprises a substrate, a proximal side of which is positioned toward the skin of the subject during use; at least one fluid receptacle positioned on the substrate and containing a store of the drug; at least one ejection chamber positioned on the proximal side of the substrate; at least one fluid via fluidly connecting the at least one fluid receptacle with the at least one ejection chamber; means for causing ejection of fluid from the at least one ejection chamber; and logic, wherein the logic is programmed to initiate ejection of an amount of fluid from the at least one ejection chamber by the means for causing ejection. In embodiments, the means for causing fluid ejection comprises at least one heater or heater array adjacent at least a portion of the at least one ejection chamber; and wherein the logic is programmed to initiate the at least one heater or heater array to cause high pressure ejection of an amount of fluid from the at least one ejection chamber either according to a timer, upon receipt of biofeedback from the subject, or a combination thereof. The method may further comprise measuring at least one biological parameter of the subject and the logic may be programmed to receive and/or utilize the measured value of the at least one biological parameter to determine the desired amount and/or timing of fluid transferred from the at least one fluid via to the at least one ejection chamber, whereby the amount of fluid introduced into the skin via ejection from the at least one ejection chamber is adjustable based on the biofeedback.

### BRIEF DESCRIPTION OF THE DRAWINGS

For a detailed description of exemplary embodiments of the invention, reference will now be made to the accompanying drawings in which:

FIG. 1 is a schematic of a device suitable for fluid delivery into the skin, muscle and/or blood of a subject according to an embodiment of this disclosure, indicating transfer of fluid from a fluid storage receptacle to an ejection chamber by way of a fluid via.

FIG. 2 is a schematic of the heater chip-type device of FIG. 1, indicating ejection of fluid from an ejection chamber in accordance with an embodiment of this disclosure.

FIG. 3 is a schematic of a subcutaneous fluid delivery device as positioned on the skin of a subject during use according to an embodiment of this disclosure.

FIG. 4 is a flow diagram of a method of subcutaneous fluid delivery according to an embodiment of this disclosure.

### NOTATION AND NOMENCLATURE

Certain terms are used throughout the following description and claims to refer to particular system components. As one skilled in the art will appreciate, companies may refer to a component by different names. This document does not intend to distinguish between components that differ in name but not function. In the following discussion and in the claims, the terms ‘including’ and ‘comprising’ are used in an open-ended fashion, and thus should be interpreted to mean ‘comprising, but not limited to . . .’. The singular forms ‘a,’ ‘an,’ and ‘the’ include plural referents unless the context clearly dictates otherwise. Thus, for example, reference to ‘a medicine’ includes reference to one or more of such materials.

The term ‘via’ is used herein to refer to a pathway passing through a substrate of the subcutaneous delivery device from a fluid storage receptacle to a proximal side of the device which will be directed toward the skin of a subject during utilization of the device.

The phrase ‘high pressure’ is used to refer to a pressure sufficient to cause penetration of the epidermis by the ejected fluid. The exact pressure will depend on the individual subject being administered the medicament or tattoo via the disclosed system and method.

Also, the term ‘couple’ or ‘couples’ is intended to mean either an indirect or direct electrical connection. Thus, if a first device couples to a second device, that connection may be through a direct electrical connection, or through an indirect electrical connection via other devices and connections.

Concentrations, amounts, and other numerical data may be presented herein in a range format. It is to be understood that such range format is used merely for convenience and brevity and should be interpreted flexibly to include not only numerical values explicitly recited as the limits of the range, but also to include all the individual numerical values or sub-ranges encompassed within that range as if each numerical value and sub-range is explicitly recited.

### DETAILED DESCRIPTION

Overview. The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

As mentioned hereinabove, conventional methods of injecting medicine into the skin include needle injection, with concomitant puncturing of the skin, and pressure gun injection utilizing a pressure gun which may be unwieldy and/or imprecisely deliver medicine. Typical non-needle devices for delivering medicine into the skin are bulky. The amount of medicine delivered into the skin using such devices is often imprecise and delivery using such devices is often painful. This disclosure provides devices and methods of delivering fluids into the skin that do not cause pain and may provide more precise delivery than conventional devices and methods.

The disclosed apparatus and method are adapted from the field of inkjet printing to deliver medicine to the skin of a subject in a similar manner to deposition of ink onto a substrate via inkjet. Inkjet printers are printers that operate by propelling variably-sized droplets of liquid or molten material (i.e., ink) onto almost any sized page. Most consumer inkjet printers use print cartridges with a series of tiny electrically heated chambers constructed by photolithography. To produce an image, the printer runs a pulse of current through the heating elements causing a sudden volume change in the chamber to form a bubble, which propels a droplet of ink onto the paper. The surface tension of the ink as well as the condensation and resulting contraction of the vapor bubble draws a further charge of ink into the chamber through a narrow channel attached to an ink reservoir. Such inkjet printers are sometimes known as bubble jet printers. A printer using the bubble-jet method generally comprises an ink jet discharging port, an ink flow path connected with the ink discharging port, and an electrothermal converting element as a means for generating energy to discharge ink provided in the ink flow path.

A printhead is the device in printers, copiers and multi-function products which sprays droplets of ink onto a sheet of paper. A number of printers, copiers and multi-function products utilize heater chips in their printheads for discharging ink drops from one or more ink vias Inkjet heater chips typically contain one or more ink vias as well as arrays of heaters located next to each ink via. The heaters provide thermal energy that causes the discharge of a droplet of ink. Chip bondpads are used to power the heaters by providing electric current to the heaters. Each chip bondpad usually has a corresponding tab circuit connection, tab trace, and electrical connection to the printing device.

Most commercial and industrial ink jet printers use a piezoelectric material in an ink-filled chamber behind each nozzle instead of a heating element. In such piezoelectric inkjet printers, when a voltage is applied, the piezoelectric material changes shape or size, thus generating a pressure pulse in the fluid and forcing a droplet of ink from the nozzle. This is essentially the same mechanism as the thermal inkjet but generates the pressure pulse using a different physical principle. Piezoelectric (also called Piezo) ink jet allows a wider variety of inks than thermal or continuous ink jet but the print heads are typically more expensive. There is a drop-on-demand or DOD process, with software that directs the heads to apply between zero to eight droplets of ink per dot and only where needed.

Another inkjet method, the continuous ink jet method, is used commercially for marking and coding of products and packages. In continuous ink jet technology, a high-pressure pump directs liquid ink from a reservoir through a gunbody and a microscopic nozzle, creating a continuous stream of ink droplets via the Plateau-Rayleigh instability. A piezoelectric crystal creates an acoustic wave as it vibrates within the gunbody, causing the stream of liquid to break into droplets at regular intervals. Tens to hundreds of thousands of drops per second may be formed via the continuous inkjet method. Continuous ink jet is one of the oldest ink jet technologies in use and is fairly mature. One of its advantages is the very high velocity of the ink droplets, which allows for a relatively long distance between the print head and the substrate. Another advantage is the absence of nozzle clogging as the jet is always in use, therefore allowing volatile solvents such as ketones and alcohols to be employed, giving the ink the ability to “bite” into the substrate and dry quickly.

In embodiments, a device comprising a heater chip (similar in concept to the heater chips utilized in inkjet printers) is utilized to inject medicine into the skin. The device may comprise a heater chip coupled to other integrated circuits (hereinafter IC's). Such devices and methods of fluid delivery utilizing such devices may provide numerous advantages relative to conventional fluid delivery devices and methods. The advantages may include, but are not limited to, (1) precision of the amount of medicine injected into the skin; (2) small size of the delivery device; (3) control of medicine injection by logic integrated together with heater or piezoelectric chip, allowing precise timing and amount of injection and subject-specific programming based on the needs of a specific subject; (4) absence of moving parts in the device, promoting reliability; (5) ease of application, allowing utilization by a subject who may not be a medical professional; and (6) low cost and/or disposable design.

Various embodiments will now be described more fully hereinafter with reference to the accompanying figures, in which like numerals indicate like elements throughout the several drawings. Some, but not all embodiments of the invention are described. Indeed, such embodiments may be implemented in many different forms and should not be construed as limited to the embodiments set forth herein; rather, these embodiments are provided so that this disclosure will satisfy applicable legal requirements.

Device for Delivery of Fluid into the Skin. Herein disclosed is a device for delivering fluid through the skin of a subject. The device comprises: a substrate; a substrate, a proximal side of which is positioned toward the skin of the subject during use; at least one fluid receptacle positioned on the substrate and containing a store of the drug; at least one ejection chamber positioned on the proximal side of the substrate; at least one fluid via fluidly connecting the at least one fluid receptacle with the at least one ejection chamber; means for causing ejection of fluid from the at least one ejection chamber; and logic, wherein the logic is programmable to initiate ejection of an amount of fluid from the at least one ejection chamber by the means for causing ejection at a pressure sufficient for penetration of the epidermis of the subject. The means for causing ejection of fluid from the at least one ejection chamber may comprise a transducer selected from the group consisting of thermoelectric, piezoelectric, electrorestrictive, magnetostrictive and electromechanical transducers. Fluidly connecting may be through a microchannel which is known in the art. At least an additional fluid may be stored in at least a second fluid receptacle. The fluids may be mixed using a mixing microchannel that connects to the at least two liquid-introducing microchannels, wherein the liquids are transported from the respective liquid-introducing microchannels toward the mixing microchannel, the apparatus further comprising means for enhancing the mixing of the liquids that converge in the mixing microchannel.

The following description will be made primarily with reference to heater chip-type devices, wherein the means for causing ejection comprises transfer of heat to a portion of the fluid in the ejection chamber. It is to be understood that various transducers, such as piezoelectric, electrorestrictive, magnetostrictive and electromechanical transducers may be utilized in various embodiments as a means for causing ejection of fluid from an ejection chamber rather than thermoelectric transducers.

In embodiments, the device is configured as a thermal inkjet-type dispenser. In embodiments, the device is configured as a piezo-electric inkjet-type dispenser. The device may comprise a heater-chip or other inkjet-type microchip adapted for subcutaneous and/or intramuscular delivery of medicine to a subject. Although suitable for intramuscular and/or subcutaneous medicine delivery, description will be made with regard to subcutaneous delivery.

In embodiments, the device is configured as a heater chip, and the means for causing ejection of fluid from the at least one ejection chamber comprises at least one heater or heater array adjacent at least a portion of the at least one ejection chamber such that, upon initiation, heat is applied to a portion of the fluid in the ejection chamber, vaporizing that portion of fluid causing it to expand rapidly and force fluid out of the ejection chamber and into the skin of the subject.

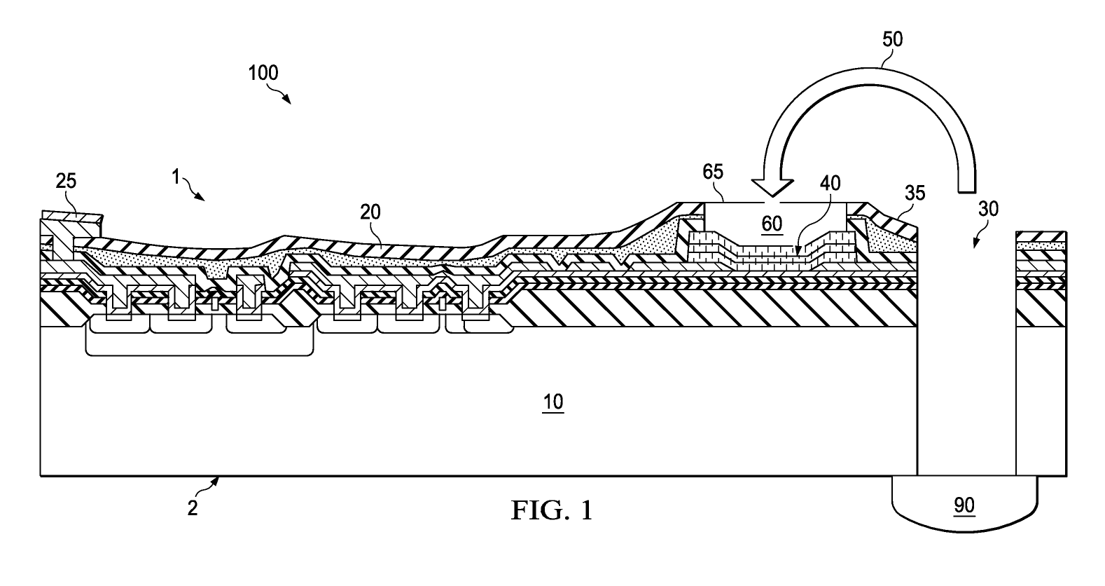

FIG. 1 is a schematic of a heater chip-type device 100 suitable for fluid delivery into the skin of a subject according to an embodiment of this disclosure. Device 100 comprises storage receptacle 90, ejection chamber 60, fluid via 30 and supply channel 35 fluidly connecting storage receptacle 90 and ejection chamber 60, means for causing ejection 40, biosensor 25 and logic 20 (e.g., CMOS), positioned on a substrate 10. The substrate of device 100 may comprise any suitable semiconductor material. The substrate 10 may be selected from ceramics, polymers, silicon, and metals. In embodiments, substrate 10 comprises silicon.

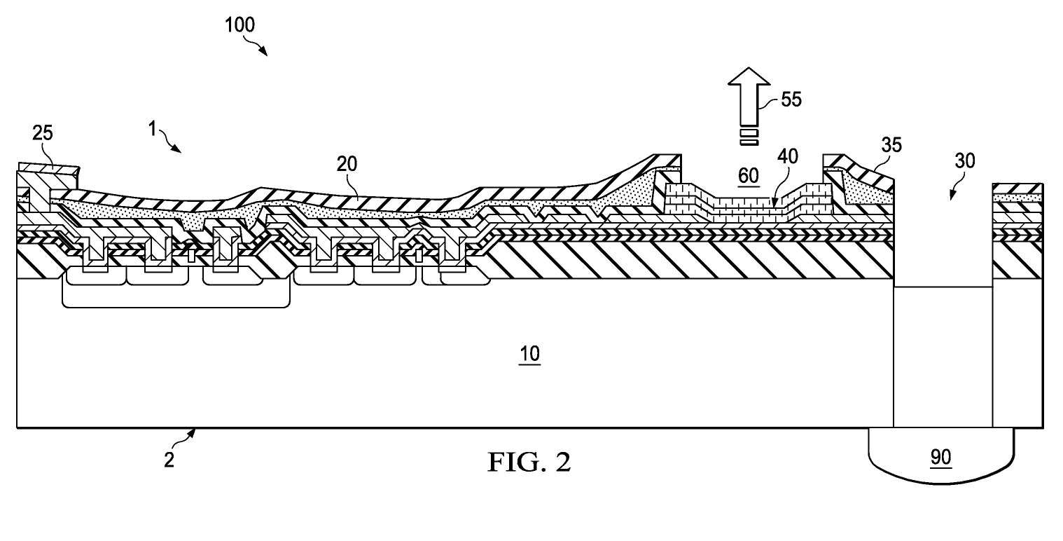

In embodiments, the means for causing ejection of a desired amount of fluid 40 is a thermal means (e.g. electrothermal means). For example, the means 40 may comprise one or more heaters or heating elements. Upon initiation by logic 20, one or more heating elements or heaters 40 rapidly (e.g., within milliseconds) heats a desired amount of fluid in ejection chamber 60, converting the fluid near the heaters therein to a gas, thus increasing the pressure at the bottom and causing expelling of the fluid on top from ejection chamber 60. The amount of fluid ejected at each initiation can be precisely controlled by the geometry and positioning of the heater(s) 40, supply channel 35, the heater temperature, the rate of heating, etc. Multiple ejections can be initialized by logic 20 to deliver a desired amount of fluid (i.e. medicine) into the skin. Initiation may cause a desired amount of fluid to be transferred from fluid via 30 to ejection chamber 60. Alternatively, ejection of fluid from ejection chamber 60 may be designed to draw an amount of fluid (for example due to surface tension and vacuum caused by expelling of fluid from ejection chamber 60) from fluid via 30 and storage receptacle 90 into ejection chamber 60 via supply channel 35, providing the fluid to be ejected at the next time of initiation by logic 20. Although indicated as a dome-shaped receptacle in FIGS. 1 and 2, storage receptacle 90 may have any suitable shape, such as rectangular.

Heater 40 is adjacent the ejection chamber and is effective for heating a portion of the fluid transferred by supply channel 35 to bubble or ejection chamber 60 from fluid via 30 as indicated at arrow 50. FIG. 2 is a schematic of heater chip 100 at such point as ejection of an amount of fluid transferred from fluid via 30 to ejection chamber 60 has been initiated by logic 20 and caused by ejection means or heater 40. As indicated in FIG. 2, heating of the portion of the fluid near heater 40 causes rapid expansion of the heated fluid and ejection of the fluid within ejection chamber 60, as indicated at 55. Heater 40 may comprise a heater array. The heater array may comprise a plurality of thin film resistors, as known in the art.

A nozzle plate 65, as known in the art, may be positioned above ejection chamber 60 and/or fluid via 30 to direct the ejected fluid toward the skin. Such a nozzle plate 65 may have one or more openings extending through it which define one or more nozzles through which droplets or bubbles of fluid are ejected. Nozzle plate 65 may be bonded to device (e.g., chip) 100 via any art recognized technique, including a thermocompression bonding process. The positioning of nozzle plate 65 may define the one or more ejection chambers or bubble chambers 60. Fluid supplied by fluid via 30 flows into bubble or ejection chamber 60 via fluid supply channel 35. For stability, in embodiments, device 100 comprises a microchip (e.g. a heater chip) supported by a base. In such instances, the bottom side the microchip may be affixed to a base material. The microchip may be affixed to the base material by any suitable means, for example, adhesive. The base material may be, for example, selected from polymers, metals, silicon, ceramics, and combinations thereof. The total thickness of the fluid delivery device 100 may be in the range of from about 400 μm to about 1 cm, in the range of from about 400 μm to about 1 mm, less than about 1 cm, less than about 1 mm, or less than about 400 μm. The thickness of the microchip may be in the range of from about 150 μm to about 2500 μm.

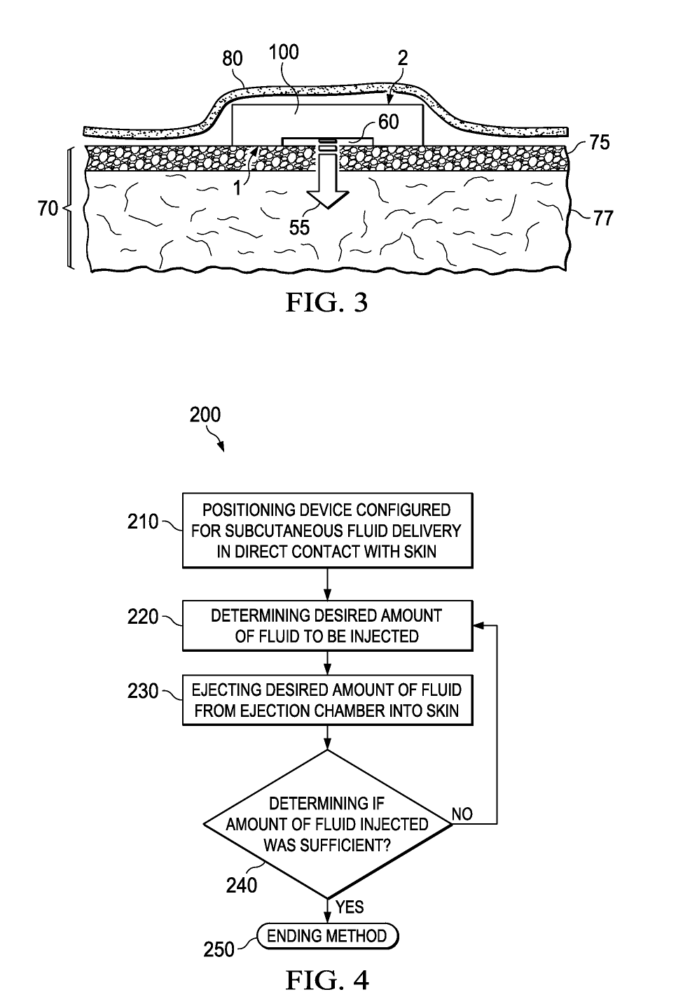

In device 100 the side of ejection chamber 60 distal heater 40 is positioned towards the skin of a subject during operation. Of course, for utilization of device 100 requiring contact with the skin of a subject, all components of device 100 which will come into contact with the skin of the subject are made of biocompatible materials. As indicated in FIG. 3, which is a schematic of device 100 positioned on the skin 70 of a subject during use according to an embodiment of this disclosure, ejection chamber 60 is positioned on proximal side 1 of the device, which will contact the skin of the subject during operation. Substrate 10 of the (heater) chip or a base upon which the microchip is supported provide distal side 2 of device 100, located away from the skin of a subject during operation. During operation, a bandage 80 may be used to position subcutaneous fluid delivery device 100 on the skin 70 of a subject such that nozzle plate 65 is directed toward the epidermis 75 of the subject. FIG. 3 also indicates introduction of fluid at arrow 55 into the dermis 77 of the subject via the subcutaneous fluid delivery device 100.

Subcutaneous fluid delivery device 100 may further comprise means 25 for sensing a biological parameter of the subject. The means for sensing may be any means known in the art for determining a biological parameter of the subject. In embodiments, the means for sensing 25 comprises one or more biosensors positioned on the subcutaneous fluid delivery device 100 such that the one or more biosensors contact the surface of the skin of the subject during use and wherein the one or more biosensors are operable to measure at least one bioparameter. The one or more biosensors may be configured for measuring body temperature of the subject, blood pressure of the subject, heart rate (i.e. pulse) of the subject, or a combination thereof. In embodiments, means for sensing 25 comprises a medical (skin) infrared thermometer which is capable of measuring the temperature of a subject when contacted with the skin. In embodiments, means for sensing comprises a skin blood pressure sensor, which is capable of determining, e.g. photoelectrically, blood pressure when in contact with the skin.

In embodiments, the means for sensing 25 comprises a receiver configured to receive the value of a biological parameter determined by apparatus outside the subcutaneous fluid delivery device 100. In embodiments, for example, the means for sensing comprises a sensor (e.g., an IC) outside subcutaneous fluid delivery device 100. The external sensor may be configured to measure/determine a biological parameter upon introduction of the external sensor into the blood of the subject, as predicated by the art. The external sensor may be a sensor configured to measure the level of at least one component in the blood of the subject. In embodiments, the at least one component is selected from the group consisting of blood sugar, medicines the subject is using, and blood gases. The external sensor may send a value of the measured value wirelessly to the means for sensing (e.g. a receiver) 25 of device 100.

In embodiments, the at least one storage receptacle 90 of subcutaneous fluid delivery device 100 contains a fluid selected from tattooing dyes and medicines. The medicine may be any medicine known to be effective when introduced into the blood, skin or muscle of a subject via the skin. By way of example, the medicine may be selected from the group consisting of blood pressure medicines, heart rate controlling medicines, medicines for altering blood sugar, antidepressants, cholesterol altering medicines, heart attack medicines, such as nitroglycerin and combinations thereof. Medicine suitable for application to the skin, such as anti-itch medicine or medicine for the treatment of sunburn may also be administered to the dermis via the disclosed device and method.

Subcutaneous fluid delivery device 100 further comprises logic. Logic 20 is programmable to initiate high pressure ejection of an amount of fluid from the at least one ejection chamber 60 by the means for causing ejection. The logic may be implemented by any suitable circuitry and transistors, as known in the art.

In embodiments, logic 20 comprises CMOS technology and utilizes metal oxide semiconductor field effect transistors or MOSFETs. The metal-oxide-semiconductor field-effect transistor (MOSFET, MOS-FET, or MOS FET) is a device used to amplify or switch electronic signals. The MOSFET includes a channel of n-type or p-type semiconductor material, and is accordingly called an NMOSFET or a PMOSFET (also commonly nMOS, pMOS). MOSFETs may be desirable, in applications, due to their utility in low-power devices, usually in the CMOS configuration. Since a CMOS device only draws current on the transition between logic states, CMOS devices consume much less current than bipolar devices, and may be desirable in the instant applications. In embodiments, logic 20 comprises digital CMOS logic, which uses p- and n-channel MOSFETs as building blocks. CMOS logic reduces power consumption because little or no current flows, and thus no power is consumed, except when the inputs to logic gates are being switched. CMOS may be desirable for use in device 100 as it may result in minimal power consumption and heat generation. In applications, logic 20 comprises analog CMOS, digital CMOS, or a combination thereof.

In embodiments, logic 20 comprises bipolar logic and utilizes bipolar junction transistors, or BJTs. In embodiments, the logic array(s) 20 comprises BiCMOS technology and utilizes both MOSFETs and BJTs.

In embodiments, logic 20 is programmed to initiate high pressure ejection of an amount of fluid from the at least one ejection chamber 60 according to a preset timer. In embodiments, the logic is operable to determine the timing and/or the amount of fluid transferred from fluid via 30 and ejected via ejection chamber 60 by the means for causing ejection 40 (e.g. by heater) based on a sensed biological parameter. The amount of medicine delivered can be determined based on a preset value or may be determined via a continuous or semi-continuous feedback signal from means for sensing 25 (e.g., via signal received by a receiver 25 on device 100 from an external biosensor or via a bioparameter measured by a sensor 25 integrated with device 100). The amount of fluid injected may be administered via single or multiple ejections from ejection chamber 60. As discussed hereinabove, the biological sensor can be a remote device which sends the value of one or more biological parameter through wire or wirelessly to a receiver which is part of logic 20 or is otherwise integrated with device 100. Alternatively, the biological sensor can be an integral part of logic 20 or a distinct device (e.g. MEMS) integrated with device 100.

Method of Subcutaneous Fluid Delivery. Also disclosed herein is a method of subcutaneous fluid delivery. One or more fluid may be delivered by utilizing one or more devices configured for delivering a single fluid or a single device configured for delivering more than one fluid. The fluid(s) to be delivered may be selected, for example, from medicines and tattoo dyes.

Description of a method of subcutaneous fluid delivery will now be made with reference to FIG. 4. FIG. 4 is a flow diagram of a method 200 of subcutaneous fluid delivery according to an embodiment of this disclosure. Method 200 comprises positioning a device configured for subcutaneous fluid delivery in direct contact with the skin of a subject 210; determining a desired amount of fluid to be injected through the skin 220; ejecting the desired amount of fluid from one or more ejection chamber 230; determining if the amount of fluid injected was sufficient 240; and ending of the method at 250. If the amount of fluid injected is determined to be insufficient at 240, steps 220, 230, and/or 240 may be repeated until the amount of fluid that was injected is sufficient, at which time the method may be terminated.

In embodiments, positioning a device configured for subcutaneous fluid delivery 210 comprises positioning a device as described hereinabove in direct contact with the skin of a subject. In embodiments, providing a device configured for subcutaneous fluid delivery 210 comprises providing a device configured and programmed for subcutaneous fluid delivery, wherein the device comprises a substrate 10; at least one fluid storage receptacle 90 containing therein a store of the fluid, at least one ejection chamber in fluid communication with the at least one storage receptacle by at least one fluid via 30 and at least one supply chamber 35, means for causing ejection of fluid from the at least one ejection chamber; and logic, wherein the logic is programmable to initiate ejection, at a pressure sufficient to cause penetration of the skin of the subject by the ejected fluid, of an amount of fluid from the at least one ejection chamber 60 by the means for causing ejection 40. Positioning the subcutaneous fluid delivery device in direct contact with the skin of a subject 210 comprises positioning the subcutaneous fluid delivery device 100 such that the side of the ejection chamber 60 distal the means for causing ejection 40 is directed toward the skin of the subject. A device 100 may be affixed to the skin of a subject by any means known in the art, for example, with a bandage, as indicated in FIG. 3. Device 100 may comprise a plurality of fluid vias 30, storage receptacles 90, ejection chambers 60, supply channels 35 and/or means for causing ejection (e.g., heaters 40) from the ejection chambers 60. In embodiments, the subcutaneous fluid delivery device comprises at least two fluid vias and/or at least two storage receptacles 90.

Method 200 further comprises determining the amount of fluid to be injected 220. The amount of fluid to be injected may be preprogrammed into logic 20 of device 100 or may be determined based upon biofeedback. As discussed above, the biofeedback may be provided by one or more external biosensors to a receiver on device 100, in which case the means for sensing 25 comprises a receiver, as discussed above. Alternatively or additionally, the biofeedback may be provided by a sensor positioned on the device itself, in which case the means for sensing 25 comprises a biosensor. For example, upon positioning of the device 210 a bioparameter measured by an external biosensor or by a biosensor positioned on the device may be received by logic 20, which is programmed to calculate an amount of fluid to be injected based upon the biofeedback. Alternatively, no biosensor(s) is (are) utilized, and the logic 20 is programmed to inject a desired amount of fluid. Logic 20 may be programmed to inject a desired amount of fluid according to a preset time schedule.

Determining the amount of fluid to be injected 220 may thus further comprise measuring at least one biological parameter of the subject. In such instances logic 20 is configured to receive the biofeedback and utilize it to determine the timing and/or the desired amount of fluid to be transferred from fluid via 30 to ejection chamber 60 and/or the amount of fluid to be ejected from ejection chamber 60 into the skin of the subject. In such embodiments, the amount of fluid introduced into the skin via ejection from the at least one ejection chamber 60 is adjustable based on the biofeedback. In embodiments, the at least one biological parameter comprises body temperature, blood pressure or heart rate of the subject. In embodiments, the at least one biological parameter is selected from the group consisting of the levels of components in the blood of the subject, the body temperature of the subject, the blood pressure of the subject, the heart rate of the subject, and combinations thereof. Components of the blood measured for use as biofeedback include, without limitation, blood glucose, blood gases, medicines the subject utilizes, cholesterol, and combinations thereof. The at least one biological parameter may be measured with at least one biosensor configured to sense the biological parameter of the subject. In applications, the at least one biosensor is positioned on the device such that it contacts the skin of the subject during use of the device. The at least one biosensor may comprise, for example, one or more selected from the group consisting of temperature sensors, blood pressure sensors and heart rate sensors. For example, the fluid to be injected may be nitroglycerin and the biosensor may measure an indicator of impending heart attack symptoms, such as elevated heart rate.

As discussed above, in embodiments, the at least one biosensor comprises an external biosensor operable to measure a bioparameter upon introduction of the biosensor (e.g., an IC) into the blood of the patient. In such instances, the external biosensor is programmed to measure the selected bioparameter(s) and wirelessly transmit the information to a receiver 25 of device 100, which may be part of logic 20. In such instances, logic 20 is programmed to utilize the received data to determine the amount of fluid to be ejected 220 from ejection chamber 60 and thus introduced into the skin of the subject.

Method 200 further comprises ejecting a desired amount of fluid from the ejection chamber into the skin 230. In embodiments, the device is configured as a heater chip and the means for causing fluid ejection 40 comprises at least one heater or heater array adjacent the at least one ejection chamber; and the logic is programmed to initiate the at least one heater or heater array to cause high pressure ejection of the desired amount of fluid from the at least one ejection chamber either at a certain time, upon receipt of biofeedback from the subject, or both. Initiation may comprise, for example, initiating transfer of a desired portion of fluid from fluid via 30 to ejection chamber 60 and/or heating of a desired portion of the fluid within ejection chamber 60 by a heater 40. A portion of the fluid transferred from the fluid via 30 to ejection chamber 60 via supply channel 35 may be heated to a temperature in the range of from about 100° C. to about 600° C., in the range of from about 400° C. to about 600° C., or less than about 600° C., 500° C., 400° C., 300° C., 200° C., or less than about 100° C. to cause rapid heating and expansion of the heated portion and resultant ejection of fluid from ejection chamber 60. Ejection may be facilitated by an appropriately designed nozzle plate 65. Desirably, the one or more heater is positioned only at the bottom of the ejection chamber such that the majority of the fluid remains liquid (and which may have a temperature less than, for example, about 100° C.). As discussed above, the ejected fluid may be forced through an ejection nozzle of nozzle plate 65 positioned above ejection chamber 60. In embodiments, a portion of the fluid transferred from the fluid via 30 to ejection chamber 60 may be heated to a temperature in the range of from about 400° C. to about 600° C. to cause ejection of fluid from ejection chamber 60. In instances when the fluid is a medicine, the device must be configured such that efficacy of the medicine is maintained during the ejection process. For example, designing the ejection chamber 60 or nozzles of nozzle plate 65 to have smaller volumes or openings, respectively, may be utilized to enhance lower temperature ejection of fluid from ejection chamber(s) 60.

Fluid is ejected from ejection chamber 60 at a pressure sufficient to cause penetration of the epidermis, such that the ejected fluid effectively enters the dermis of the subject. Referring now to FIG. 1, ejecting the desired amount of fluid from the ejection chamber 60 into the skin comprises logic 20 initiating heater 40 whereupon the fluid at the end of ejection chamber 60 proximal the heater is heated and vaporizes. The expansion of the heated fluid forces the liquid at the distal end of ejection chamber to forcefully eject from ejection chamber 60 and penetrate the epidermis of the subject. Following ejection, logic 20 terminates heating by heater 40, whereupon fluid is drawn, due to surface tension and/or pressure difference, from storage receptacle 90, via 30, and supply channel 35 into ejection chamber 60, effectively reloading ejection chamber 60 for a subsequent ejection.

Method 200 further comprises determining if the amount of fluid injected was sufficient 240. In embodiments, logic 20 is programmed to determine if the amount of fluid injected at 230 was sufficient. The determination may be based on a feedback parameter measured, via one or more biosensors, subsequent injection at 230 or may be determined to be sufficient due to original programming of logic 20 to deliver a set amount of fluid. If the amount of fluid injected at 230 is determined at 240 to be sufficient, the method is terminated 250 for the time being (if the logic is programmed to inject according to a time schedule) or permanently. If the amount of fluid ejected at 230 is determined at 240 to be insufficient, steps 220, 230, and 240 may be repeated until such time as the amount of fluid ejected is determined at 240 to have been sufficient, at which time the method is ended 250.

In embodiments, the method is utilized to subcutaneously deliver one or more medicines to the skin (dermis and/or epidermis), muscle or blood of the subject. The one or more medicines may be selected, for example, from insulin, sugar solutions, antidepressants, cholesterol medicines, high blood pressure medicines, and combinations thereof. In embodiments, the method is utilized to deliver appropriate amounts of opposing medications into the blood of the subject, for example, blood glucose reducers and blood glucose elevators. In applications in which multiple fluids are potentially to be delivered, for example, where a medicine suitable for increasing blood glucose (e.g., sugar solution) or a medicine effective for decreasing blood glucose (i.e., insulin) are to be delivered depending on the blood glucose level measured as biofeedback, two separate subcutaneous fluid delivery devices may be utilized (one comprising a storage receptacle containing insulin and logic programmed to inject upon a high blood glucose biofeedback; and a second device comprising a storage receptacle containing a blood glucose increaser such as sugar solution and logic programmed to initiate ejection of a desired amount of fluid from the storage receptacle upon a low blood glucose biofeedback). Alternatively, a single subcutaneous fluid delivery device may be utilized, the single subcutaneous fluid delivery device comprising two fluid receptacles, one containing one medicine (e.g. insulin) and the other containing another medicine (e.g. blood sugar elevator), and logic programmed to initiate ejection of a desired amount of fluid from the appropriate storage receptacle based on the biofeedback (e.g., the measured blood glucose level).

The method of subcutaneous fluid delivery may be utilized to form a tattoo on the skin of the subject. In such applications, the subcutaneous delivery device comprises one or more fluid receptacles and vias, each fluid receptacle comprising a tattoo dye, and the logic programmed to initiate ejection of desired amounts of fluid from the one or more fluid receptacle(s) such that a desired pattern is introduced into the skin of the subject.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A device configured for drug delivery through the skin of a subject, the device comprising:
a substrate, a proximal side of which is positioned toward the skin of the subject during use;
at least one fluid receptacle positioned on the substrate and containing a store of the drug;
at least one ejection chamber positioned on the proximal side of the substrate;
at least one fluid via fluidly connecting the at least one fluid receptacle with the at least one ejection chamber;
a heating element positioned adjacent at least a portion of the at least one ejection chamber, said heating element configured to rapidly heat and vaporize the fluid along that portion of the ejection chamber, causing ejection of fluid therefrom; and
logic, wherein the logic is programmable to initiate heating by the heating element, whereby an amount of fluid is ejected from the at least one ejection chamber at a pressure sufficient to penetrate the epidermis of the subject.

2. The device of claim 1 wherein the logic is programmed to initiate high pressure ejection of an amount of fluid from the at least one ejection chamber according to a preset timer.

3. The device of claim 1 wherein the substrate comprises silicon.

4. The device of claim 1 further comprising means for sensing a biological parameter of the subject and wherein the logic is operable to determine the timing of fluid ejection, the amount of fluid ejection, or both from the at least one ejection chamber based on the sensed biological parameter.

5. The device of claim 4 wherein the means for sensing comprises a biosensor positioned on the device such that it contacts the surface of the skin of the subject during use.

6. The device of claim 5 wherein biological parameter is selected from the group consisting of the body temperature of the subject, the heart rate of the subject, the blood pressure of the subject, and combinations thereof.

7. The device of claim 4 wherein the means for sensing comprises a receiver for the value of a biological parameter determined outside the device.

8. The device of claim 7 wherein the value of the biological parameter is measured by an external biosensor located outside the device.

9. The device of claim 8 wherein the external biosensor is configured to determine a biological parameter upon introduction into the blood of the subject.

10. The device of claim 9 wherein the external biosensor is configured to measure the level of at least one component in the blood of the subject.

11. The device of claim 10 wherein the external biosensor is configured to measure at least one component selected from the group consisting of blood sugar, a medicine the subject is using, and a blood gas.

12. The device of claim 1 wherein the drug is selected from the group consisting of insulin, sugar solutions, antidepressants, cholesterol medicines, high blood pressure medicines, and combinations thereof.

13. A device configured for delivery of fluid through the skin of a subject, the device comprising:
a substrate, a proximal side of which is positioned toward the skin of the subject during use;
at least one fluid receptacle positioned on the substrate and containing a store of the fluid;
at least one ejection chamber positioned on the proximal side of the substrate;
at least one fluid via fluidly connecting the at least one fluid receptacle with the at least one ejection chamber;
means for causing ejection of fluid from the at least one ejection chamber; and
logic, wherein the logic is programmed to initiate high pressure ejection of an amount of fluid from the at least one ejection chamber by the means for causing ejection, the high pressure ejection at a pressure sufficient to penetrate the epidermis of the subject.

14. The device of claim 13 wherein the means for causing ejection of fluid from the at least one ejection chamber comprises at least one heater or heater array adjacent at least a portion of the at least one ejection chamber.

15. The device of claim 14 wherein the at least one heater array comprises a plurality of thin film resistors.

16. The device of claim 13 wherein the means for causing ejection of fluid from the at least one ejection chamber comprises a transducer selected from the group consisting of piezoelectric, electrorestrictive, magnetostrictive and electromechanical transducers.

17. The device of claim 13 wherein the fluid is selected from medicines and tattoo dyes.

18. A method of introducing a fluid into the skin of a subject, the method comprising:
positioning a device configured and programmed for subcutaneous fluid delivery in direct contact with the skin of the subject, wherein the device comprises a substrate, a proximal side of which is positioned toward the skin of the subject during use; at least one fluid receptacle positioned on the substrate and containing a store of the drug; at least one ejection chamber positioned on the proximal side of the substrate; at least one fluid via fluidly connecting the at least one fluid receptacle with the at least one ejection chamber; means for causing ejection of fluid from the at least one ejection chamber; and logic, wherein the logic is programmed to initiate ejection of an amount of fluid from the at least one ejection chamber by the means for causing ejection.

19. The method of claim 18 wherein the means for causing fluid ejection comprises at least one heater or heater array adjacent at least a portion of the at least one ejection chamber; and wherein the logic is programmed to initiate the at least one heater or heater array to cause high pressure ejection of an amount of fluid from the at least one ejection chamber either according to a timer, upon receipt of biofeedback from the subject, or a combination thereof.

20. The method of claim 18 further comprising measuring at least one biological parameter of the subject and wherein the logic is programmed to receive and/or utilize the measured value of the at least one biological parameter to determine the desired amount and/or timing of fluid transferred from the at least one fluid via to the at least one ejection chamber, whereby the amount of fluid introduced into the skin via ejection from the at least one ejection chamber is adjustable based on the biofeedback.
